(19) United States
(12) Patent Application Publication
(10) Pub. No.: US 2021/0002707 A1
(43) Pub. Date: Jan. 7, 2021

(54) NANOVESICLES DERIVED FROM BLAUTIA BACTERIA AND USE THEREOF

(71) Applicant: MD HEALTHCARE INC., Seoul (KR)

(72) Inventor: Yoon-Keun KIM, Paju-Si, Gyeonggi-do (KR)

(73) Assignee: MD HEALTHCARE INC., Seoul (KR)

(21) Appl. No.: 16/975,890

(22) PCT Filed: Feb. 21, 2019
(86) PCT No.: PCT/KR2019/002102

§ 371 Date: Aug. 26, 2020

(30) Foreign Application Priority Data
Feb. 26, 2018 (KR) 10-2018-0023210
Feb. 20, 2019 (KR) 10-2019-0019742

(51) Int. Cl. C12Q 1/689; A61K 35/74; A23L 33/135

(52) U.S. Cl. CPC C12Q 1/689; A23V 2002/00; A23L 33/135; A61K 35/74

(57) ABSTRACT

Provided are vesicles derived from bacteria of the genus Blautia and a use thereof for diagnosing colon cancer, liver cancer, pancreatic cancer, cholangiocarcinoma, ovarian cancer, bladder cancer, myocardial infarction, atrial fibrillation or variant angina pectoris, and for developing a composition for preventing, alleviating or treating a colorectal disease, a liver disease, a pancreatic disease, cholangiocarcinoma, ovarian cancer, bladder cancer, myocardial infarction, atrial fibrillation, variant angina pectoris or a graft-versus-host disease.

### TECHNICAL FIELD

The present invention relates to nanovesicles derived from bacteria of the genus Blautia and a use thereof, and more particularly, to a method of diagnosing colon cancer, liver cancer, pancreatic cancer, cholangiocarcinoma, ovarian cancer, bladder cancer, myocardial infarction, atrial fibrillation or variant angina pectoris using nanovesicles derived from bacteria of the genus Blautia, and a composition for preventing, alleviating or treating the disease, which comprises the vesicle.

### BACKGROUND ART

Since the beginning of the 21st century, acute infectious diseases recognized as epidemic diseases in the past have become less important, whereas chronic inflammatory diseases accompanied by immune dysfunction caused by disharmony between humans and microbiomes have changed disease patterns as main diseases that determine the quality of life and the human lifespan. As an intractable chronic disease in the 21st century, cancer, cardiovascular diseases, chronic lung diseases, metabolic diseases, and neuropsychiatric diseases have become a big problem for public health in the country as main diseases that determine the human lifespan and the quality of life, and the intractable chronic diseases are characterized by chronic inflammation accompanying abnormal immune functions caused by causative factors.

Meanwhile, it is known that the number of microorganisms coexisting in the human body has reached 100 trillion, which is 10 times more than the number of human cells, and the number of microorganism genes is more than 100 times the number of human genes. A microbiota or microbiome refers to a microbial community including bacteria, archaea and eukarya present in a given habitat. Bacteria coexisting in our body and bacteria present in the ambient environment secrete nanometer-sized vesicles in order to exchange information on genes, low molecular compounds, proteins, and the like with other cells. The mucosa forms a physical defense membrane through which particles having a size of 200 nanometers (nm) or more cannot pass, so that bacteria coexisting in the mucosa cannot pass through the mucosa, but vesicles derived from bacteria have a size of 100 nanometers or less and are absorbed into our bodies after relatively freely passing through epithelial cells via the mucosa. Bacteria-derived vesicles that are locally secreted from bacteria are absorbed via epithelial cells of the mucous membrane to thereby induce a local inflammatory response, and the vesicles having passed through the epithelial cells are systematically absorbed via lymphatic vessels and thereby distributed in respective organs, and immune and inflammatory responses are regulated in the organs in which the vesicles are distributed. For example, vesicles derived from pathogenic gram-negative bacteria such as Escherichia coli locally cause colitis, and promote a systemic inflammatory response, and blood coagulation through a vascular endothelial inflammatory response when absorbed into blood vessels, and cause insulin resistance and diabetes when absorbed into insulin-acting muscle cells. On the other hand, vesicles derived from beneficial bacteria may control a disease by controlling immune dysfunction and metabolic dysfunction caused by pathogenic vesicles.

As immune responses to factors such as bacteria-derived vesicles, Th17 immune responses characterized by the secretion of the interleukin (hereinafter, IL)-17 cytokine occur, and IL-6 is secreted when exposed to bacteria-derived vesicles, thereby inducing Th17 immune responses. Inflammation caused by the Th17 immune response is characterized by neutrophil infiltration, and during the process by which inflammation occurs, tumor necrosis factor-alpha (hereinafter, TNF-α) secreted from inflammatory cells such as macrophages plays an important role.

Bacteria of the genus Blautia are anaerobic gram-positive bacteria found in the intestines, and were reported to reduce mortality by a graft-versus-host disease when the bacteria increase in the intestines after bone marrow transplantation. However, so far, it has not been reported that Blautia bacteria extracellularly secrete vesicles, and particularly, no cases of applying the vesicles to the diagnosis and treatment of an intractable disease such as cancer or a cardiovascular disease have been reported.

### DISCLOSURE

### Technical Problem

As a result of conducting earnest research to solve the above conventional problems, the inventors confirmed that a content of vesicles derived from bacteria of the genus Blautia is significantly decreased in a sample derived from a patient with colon cancer, liver cancer, pancreatic cancer, cholangiocarcinoma, ovarian cancer, bladder cancer, myocardial infarction, atrial fibrillation or variant angina pectoris, compared with a normal individual, through metagenomic analysis. In addition, it was confirmed that, when vesicles were isolated from Blautia coccoides included in bacteria of the genus Blautia to treat macrophages, IL-6 and TNF-α secretion caused by pathogenic vesicles was significantly inhibited. Thus, the present invention was completed.

Thus, an object of the present invention is to provide a method of providing information for diagnosis of colon cancer, liver cancer, pancreatic cancer, cholangiocarcinoma, ovarian cancer, bladder cancer, myocardial infarction, atrial fibrillation or variant angina pectoris.

In addition, the present invention is also directed to providing a composition for preventing, alleviating or treating a colorectal disease, a liver disease, a pancreatic disease, cholangiocarcinoma, ovarian cancer, bladder cancer, myocardial infarction, atrial fibrillation, variant angina pectoris or a graft-versus-host disease, which comprises vesicles derived from bacteria of the genus Blautia as an active ingredient.

However, a technical problem to be achieved by the present invention is not limited to the aforementioned problems, and the other problems that are not mentioned may be clearly understood by a person skilled in the art from the following description.

### Technical Solution

To achieve the object of the present invention as described above, the present invention provides a method of providing information for diagnosing colon cancer, liver cancer, pancreatic cancer, cholangiocarcinoma, ovarian cancer, bladder cancer, myocardial infarction, atrial fibrillation or variant angina pectoris, the method comprising the following steps:

(a) extracting DNAs from extracellular vesicles isolated from samples of a normal individual and a subject;

(b) performing polymerase chain reaction (PCR) on the extracted DNA using a pair of primers prepared based on a gene sequence present in 16S rDNA to obtain each PCR product; and

(c) classifying a case in which a content of extracellular vesicles derived from bacteria of the genus Blautia is lower than that of the normal individual sample, as colon cancer, liver cancer, pancreatic cancer, cholangiocarcinoma, ovarian cancer, bladder cancer, myocardial infarction, atrial fibrillation or variant angina pectoris, through quantitative analysis of the PCR product.

In addition, the present invention provides a method of diagnosing colon cancer, liver cancer, pancreatic cancer, cholangiocarcinoma, ovarian cancer, bladder cancer, myocardial infarction, atrial fibrillation or variant angina pectoris, the method comprising the following steps:

(a) extracting DNAs from extracellular vesicles isolated from samples of a normal individual and a subject;

(b) performing polymerase chain reaction (PCR) on the extracted DNA using a pair of primers prepared based on a gene sequence present in 16S rDNA to obtain each PCR product; and

(c) determining a case in which a content of extracellular vesicles derived from bacteria of the genus Blautia is lower than that of the normal individual sample, as colon cancer, liver cancer, pancreatic cancer, cholangiocarcinoma, ovarian cancer, bladder cancer, myocardial infarction, atrial fibrillation or variant angina pectoris, through quantitative analysis of the PCR product.

As an embodiment of the present invention, the sample in Step (a) may be blood or stool.

As another embodiment of the present invention, the primer pair in Step (b) may be primers of SEQ ID Nos. 1 and 2.

Further, the present invention provides a pharmaceutical composition for preventing or treating a colorectal disease, a liver disease, a pancreatic disease, cholangiocarcinoma, ovarian cancer, bladder cancer, myocardial infarction, atrial fibrillation, variant angina pectoris or a graft-versus-host disease, comprising vesicles derived from bacteria of the genus Blautia as an active ingredient.

Further, the present invention provides a food composition for preventing or alleviating a colorectal disease, a liver disease, a pancreatic disease, cholangiocarcinoma, ovarian cancer, bladder cancer, myocardial infarction, atrial fibrillation, variant angina pectoris or a graft-versus-host disease, comprising vesicles derived from bacteria of the genus Blautia as an active ingredient.

Further, the present invention provides a method of preventing or treating a colorectal disease, a liver disease, a pancreatic disease, cholangiocarcinoma, ovarian cancer, bladder cancer, myocardial infarction, atrial fibrillation, variant angina pectoris or a graft-versus-host disease, the method comprising a step of administering a pharmaceutical composition comprising vesicles derived from bacteria of the genus Blautia as an active ingredient to a subject.

Further, the present invention provides a use of vesicles derived from bacteria of the genus Blautia for preventing or treating a colorectal disease, a liver disease, a pancreatic disease, cholangiocarcinoma, ovarian cancer, bladder cancer, myocardial infarction, atrial fibrillation, variant angina pectoris or a graft-versus-host disease.

In one embodiment of the present invention, the vesicles may have an average diameter of 10 to 200 nm.

In another embodiment of the present invention, the vesicles may be secreted naturally or artificially from bacteria of the genus Blautia.

In another embodiment of the present invention, the vesicles derived from bacteria of the genus Blautia may be vesicles derived from Blautia coccoides.

In another embodiment of the present invention, the colorectal disease may be colitis, irritable bowel syndrome or colon cancer.

In another embodiment of the present invention, the liver disease may be hepatitis, liver cirrhosis or liver cancer.

In another embodiment of the present invention, the pancreatic disease may be pancreatitis or pancreatic cancer.

### Advantageous Effects

The inventors confirmed that intestinal bacteria are not absorbed into the body through epithelial cells, but bacteria-derived vesicles are absorbed, systemically distributed and then excreted out of the body through the kidneys, liver and lungs, and by metagenomic analysis for vesicles derived from bacteria present in patients' blood, also confirmed that vesicles derived from bacteria of the genus Blautia, which are present in blood or stool of patients with colon cancer, liver cancer, pancreatic cancer, cholangiocarcinoma, ovarian cancer, bladder cancer, myocardial infarction, atrial fibrillation, and variant angina pectoris significantly decrease, compared with a normal individual. In addition, when Blautia coccoides, which is one species of bacteria of the genus Blautia, was cultured in vitro to isolate vesicles, and then the vesicles were administered to inflammatory cells, it was confirmed that the secretion of inflammation mediators, mediated by pathogenic vesicles was significantly inhibited. Therefore, it is expected that the vesicles derived from bacteria of the genus Blautia according to the present invention can be effectively used for a method of diagnosing colon cancer, liver cancer, pancreatic cancer, cholangiocarcinoma, ovarian cancer, bladder cancer, myocardial infarction, atrial fibrillation, or variant angina pectoris, and a composition for preventing, alleviating or treating the diseases.

### DESCRIPTION OF DRAWINGS

FIG. 1A is a series of photographs capturing distribution patterns of bacteria and bacteria-derived vesicles (EV) by time after the bacteria and the vesicles derived from bacteria were orally administered to mice, and FIG. 1B is a result of evaluating the in vivo distribution patterns of the bacteria and the vesicles by harvesting blood, kidneys, liver, and various organs at 12 hours after orally administering the bacteria and the vesicles.

FIG. 2 is a result of comparing the distributions of vesicles derived from bacteria of the genus Blautia after metagenomic analysis of bacteria-derived vesicles present in the stool of colon cancer patients and a normal individual.

FIG. 3 is a result of comparing the distributions of vesicles derived from bacteria of the genus Blautia after metagenomic analysis of bacteria-derived vesicles present in the blood of liver cancer patients and a normal individual.

FIG. 3 is a result of comparing the distributions of vesicles derived from bacteria of the genus Blautia after metagenomic analysis of bacteria-derived vesicles present in the blood of liver cancer patients and a normal individual.

FIG. 4 is a result of comparing the distributions of vesicles derived from bacteria of the genus Blautia after metagenomic analysis of bacteria-derived vesicles present in the blood of pancreatic cancer patients and a normal individual.

FIG. 5 is a result of comparing the distributions of vesicles derived from bacteria of the genus Blautia after metagenomic analysis of bacteria-derived vesicles present in the blood of cholangiocarcinoma patients and a normal individual.

FIG. 6 is a result of comparing the distributions of vesicles derived from bacteria of the genus Blautia after metagenomic analysis of bacteria-derived vesicles present in the blood of ovarian cancer patients and a normal individual.

FIG. 7 is a result of comparing the distributions of vesicles derived from bacteria of the genus Blautia after metagenomic analysis of bacteria-derived vesicles present in the blood of bladder cancer patients and a normal individual.

FIG. 8 is a result of comparing the distributions of vesicles derived from bacteria of the genus Blautia after metagenomic analysis of bacteria-derived vesicles present in the blood of myocardial infarction patients and a normal individual.

FIG. 9 is a result of comparing the distributions of vesicles derived from bacteria of the genus Blautia after metagenomic analysis of bacteria-derived vesicles present in the blood of atrial fibrillation patients and a normal individual.

FIG. 10 is a result of comparing the distributions of vesicles derived from bacteria of the genus Blautia after metagenomic analysis of bacteria-derived vesicles present in the blood of variant angina pectoris patients and a normal individual.

FIG. 11 is a result of comparing degrees of secretion of inflammation mediators such as IL-6 and TNF-α with pathogenic vesicles such as E. coli EVs by treating macrophages (Raw264.7 cells) with Blautia coccoides-derived vesicles to evaluate an inflammation-inducing effect of Blautia coccoides-derived vesicles.

FIG. 12 is a result of evaluating an effect on the secretion of IL-6 and TNF-α which inflammatory mediators caused by E. coli EVs by pretreating bacteria of the genus Blautia-derived vesicles before treatment of pathogenic vesicles such as E. coli EVs to evaluate anti-inflammatory and immunomodulatory effects of Blautia coccoides-derived vesicles

### BEST MODES

The present invention relates to vesicles derived from bacteria of the genus Blautia and a use thereof.

The present inventors confirmed through metagenomic analysis that vesicles derived from bacteria of the genus Blautia in a clinical sample obtained from a patient with colon cancer, liver cancer, pancreatic cancer, cholangiocarcinoma, ovarian cancer, bladder cancer, myocardial infarction, atrial fibrillation, and variant angina pectoris, compared with a normal individual, were significantly decreased, and thus the disease can be diagnosed. In addition, as a result of isolating vesicles from Blautia coccoides, which is belonging to bacteria of the genus Blautia and analyzing their characteristics, it was confirmed that the vesicles can be used for a composition for preventing, alleviating, or treating a colorectal disease, a liver disease, a pancreatic disease, cholangiocarcinoma, ovarian cancer, bladder cancer, myocardial infarction, atrial fibrillation, variant angina pectoris or a graft-versus-host disease.

Thus, the present invention provides a method of providing information for diagnosing colon cancer, liver cancer, pancreatic cancer, cholangiocarcinoma, ovarian cancer, bladder cancer, myocardial infarction, atrial fibrillation or variant angina pectoris, the method comprising the following steps:

(a) extracting DNAs from extracellular vesicles isolated from samples of a normal individual and a subject;

(b) performing polymerase chain reaction (PCR) on the extracted DNA using a pair of primers prepared based on a gene sequence present in 16S rDNA to obtain each PCR product; and

(c) classifying a case in which a content of extracellular vesicles derived from bacteria of the genus Blautia is lower than that of the normal individual sample, as colon cancer, liver cancer, pancreatic cancer, cholangiocarcinoma, ovarian cancer, bladder cancer, myocardial infarction, atrial fibrillation or variant angina pectoris, through quantitative analysis of the PCR product.

The term “diagnosis” as used herein refers to determination of a condition of a disease of a patient over all aspects, in a broad sense. The contents of the determination are the disease entity, the etiology, the pathogenesis, the severity, the detailed aspects of a disease, the presence and absence of complications, the prognosis, and the like. The diagnosis in the present invention means determining whether colon cancer, liver cancer, pancreatic cancer, cholangiocarcinoma, ovarian cancer, bladder cancer, myocardial infarction, atrial fibrillation and/or variant angina pectoris occur, the level of the disease, and the like.

The term “nanovesicle” or “vesicle” as used herein refers to a structure consisting of a nano-sized membrane secreted from various bacteria. Vesicles derived from gram-negative bacteria or outer membrane vesicles (OMVs) have endotoxins (lipopolysaccharides), toxic protein, bacterial DNA and RNA, and vesicles derived from gram-positive bacteria also have peptidoglycan and lipoteichoic acid which are cell wall components of bacteria in addition to proteins and nucleic acids. In the present invention, nanovesicles or vesicles are secreted naturally from bacteria of the genus Blautia or produced artificially, are in the form of a sphere, and have an average diameter of 10 to 200 nm.

The term “metagenome” as used herein also refers to a microbiome, and refers to a total of genomes including all viruses, bacteria, fungi, and the like in an isolated region such as soil and an animal's intestines, and is typically used as a concept of genomes explaining identification of a large number of microorganisms at one time by using a sequence analyzer in order to analyze uncultivated microorganisms. In particular, the metagenome does not refer to a genome of one species, but refers to a kind of mixed genome as a genome of all species of one environmental unit. The metagenome is, when one species is defined in the development process of omics biology, a term derived from the viewpoint of making a complete species is made by various species interacting with each other as well as one kind of functionally existing species. Technically, the metagenome is an object of a technique to identify all species in one environment and investigate interactions and metabolism by analyzing all DNAs and RNAs regardless of species using a rapid sequence analysis method.

In the present invention, the sample derived from a subject may be blood or stool, but is not limited thereto.

Another aspect of the present invention provides a composition for preventing, treating or alleviating a malignant disease such as colon cancer, pancreatic cancer, cholangiocarcinoma, ovarian cancer, bladder cancer or lymphoma, a cardiovascular disease such as myocardial infarction, atrial fibrillation, variant angina or stroke, diabetes, Parkinson's disease, and depression, which comprises vesicles derived from bacteria of the genus Blautia as an active ingredient. The composition includes a food composition and a pharmaceutical composition, and in the present invention, the food composition includes a health functional food composition. The composition of the present invention may be prepared in a dosage form of an injection, an oral spray, or an inhalant.

In the present invention, the colorectal disease may be colitis, irritable bowel syndrome or colon cancer, but the present invention is not limited thereto.

In the present invention, the liver disease may be hepatitis, liver cirrhosis or liver cancer, but the present invention is not limited thereto.

In the present invention, the pancreatic disease may be pancreatitis or pancreatic cancer, but the present invention is not limited thereto.

The term “prevention” as used herein refers to all actions that suppress a colorectal disease, a liver disease, a pancreatic disease, cholangiocarcinoma, ovarian cancer, bladder cancer, myocardial infarction, atrial fibrillation, variant angina pectoris and/or a graft-versus-host disease or delay the onset thereof via administration of the composition according to the present invention.

The term “treatment” as used herein refers to all actions that alleviate or beneficially change symptoms of a colorectal disease, a liver disease, a pancreatic disease, cholangiocarcinoma, ovarian cancer, bladder cancer, myocardial infarction, atrial fibrillation, variant angina pectoris and/or a graft-versus-host disease via administration of composition according to the present invention.

The term “alleviation” used as used herein refers to all actions that at least reduce a parameter associated with a condition to be treated, for example, the degree of symptoms.

The vesicles may be isolated from a culturing solution comprising bacteria of the genus Blautia by using one or more methods selected from the group consisting of centrifugation, ultra-high speed centrifugation, high pressure treatment, extrusion, sonication, cell lysis, homogenization, freezing-thawing, electroporation, mechanical decomposition, chemical treatment, filtration by a filter, gel filtration chromatography, free-flow electrophoresis, and capillary electrophoresis. Further, a process such as washing for removing impurities and concentration of obtained vesicles may be further included.

The pharmaceutical composition of the present invention may include a pharmaceutically acceptable carrier. The pharmaceutically acceptable carrier is typically used in formulation, and includes saline, sterile water, Ringer's solution, buffered saline, cyclodextrin, a dextrose solution, a maltodextrin solution, glycerol, ethanol, liposomes, and the like, but is not limited thereto, and may further include other typical additives such as an antioxidant and a buffer, if necessary. Further, the composition may be formulated into an injectable formulation, such as an aqueous solution, a suspension, and an emulsion, a pill, a capsule, a granule, or a tablet by additionally adding a diluent, a dispersant, a surfactant, a binder, a lubricant, and the like. With regard to suitable pharmaceutically acceptable carriers and formulations, the composition may be preferably formulated according to each ingredient by using the method disclosed in the Remington's literature. The pharmaceutical composition of the present invention is not particularly limited in formulation, but may be formulated into an injection, an inhalant, an external preparation for skin, an oral ingestion, or the like.

The pharmaceutical composition of the present invention may be administered orally or parenterally (e.g., intravenously, subcutaneously, intradermally, intranasally or intratracheally) according to a desired method, and a dose may vary according to the condition and body weight of a patient, the severity of a disease, a drug formulation, an administration route, and duration, but may be suitably selected by those of ordinary skill in the art.

The pharmaceutical composition according to the present invention is administered in a pharmaceutically effective amount. In the present invention, the pharmaceutically effective amount refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields. The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

Specifically, an effective amount of the pharmaceutical composition according to the present invention may vary according to a patient's age, gender and body weight, and generally, the pharmaceutical composition may be administered at 0.001 to 150 mg, and preferably, 0.01 to 100 mg per kg of body weight daily or every two days, or 1 to 3 times daily. However, as the dose may be increased or decreased by an administration route, the severity of obesity, gender, a body weight or an age, the above-mentioned dose does not limit the scope of the present invention in any way.

The food composition of the present invention includes a health functional food composition. The food composition according to the present invention may be used by adding an active ingredient as is to food or may be used together with other foods or food ingredients, but may be appropriately used according to a typical method. The mixed amount of the active ingredient may be suitably determined depending on the purpose of use thereof (for prevention or alleviation). In general, when a food or beverage is prepared, the composition of the present invention is added in an amount of 15 wt % or less, preferably 10 wt % or less based on the raw materials. However, for long-term intake for the purpose of health and hygiene or for the purpose of health control, the amount may be less than the above-mentioned range.

Other ingredients are not particularly limited, except that the food composition of the present invention contains the active ingredient as an essential ingredient at the indicated ratio, and the food composition of the present invention may contain various flavorants, natural carbohydrates, and the like, like a typical beverage, as an additional ingredient. Examples of the above-described natural carbohydrate include common sugars such as monosaccharides, for example, glucose, fructose and the like; disaccharides, for example, maltose, sucrose and the like; and polysaccharides, for example, dextrin, cyclodextrin and the like, and sugar alcohols such as xylitol, sorbitol, and erythritol. As the flavorant other than those described above, a natural flavorant (thaumatin, stevia extract, for example, rebaudioside A, glycyrrhizin and the like), and a synthetic flavorant (saccharin, aspartame and the like) may be advantageously used. The proportion of the natural carbohydrate may be appropriately determined by the choice of those of ordinary skill in the art.

The food composition of the present invention may contain various nutrients, vitamins, minerals (electrolytes), flavoring agents such as synthetic flavoring agents and natural flavoring agents, colorants and fillers (cheese, chocolate, and the like), pectic acid and salts thereof, alginic acid and salts thereof, organic acids, protective colloid thickeners, pH adjusting agents, stabilizers, preservatives, glycerin, alcohols, carbonating agents used in a carbonated beverage, or the like, in addition to the additives. These ingredients may be used either alone or in combinations thereof. The ratio of these additives may also be appropriately selected by those of ordinary skill in the art.

In one embodiment of the present invention, as a result of orally administering bacteria and bacteria-derived vesicles to mice and observing in vivo absorption, distribution, and excretion patterns of the bacteria and the vesicles, it was confirmed that, while the bacteria were not absorbed via the intestinal mucous membrane, the bacteria-derived vesicles were absorbed within 5 minutes after administration and systemically distributed, and excreted via the kidneys, liver, and the like (see Example 1).

In another embodiment of the present invention, a bacterial metagenomic analysis was performed by using vesicles isolated from the blood or stool of normal individuals who were matched in age and sex with patients with colon cancer, liver cancer, pancreatic cancer, cholangiocarcinoma, ovarian cancer, bladder cancer, myocardial infarction, atrial fibrillation and variant angina pectoris. As a result, it was confirmed that vesicles derived from bacteria of the genus Blautia were significantly decreased in clinical samples of patients with colon cancer, liver cancer, pancreatic cancer, cholangiocarcinoma, ovarian cancer, bladder cancer, myocardial infarction, atrial fibrillation, and variant angina pectoris as compared to samples of normal individuals (see Examples 3 to 11).

In another embodiment of the present invention, Blautia coccoides strain was cultured to evaluate whether vesicles secreted therefrom have immunomodulatory and anti-inflammatory effects, and it was confirmed that IL-6 and TNF-α secretion caused by Escherichia coli-derived vesicles (E. coli EVs) are effectively inhibited by Blautia coccoides-derived vesicles through evaluation of the secretion of inflammatory mediators by treating E. coli EVs, which are an inflammatory disease causative factor, following treatment of macrophages with various concentrations of Blautia coccoides-derived vesicles (see Example 13).

Hereinafter, preferred Examples for helping the understanding of the present invention will be suggested. However, the following Examples are provided only to more easily understand the present invention, and the contents of the present invention are not limited by the following Examples.

### Modes of the Invention

### EXAMPLES

### Example 1. Analysis of In Vivo Absorption, Distribution, and Excretion Patterns of Intestinal Bacteria and Vesicles Derived from Bacteria

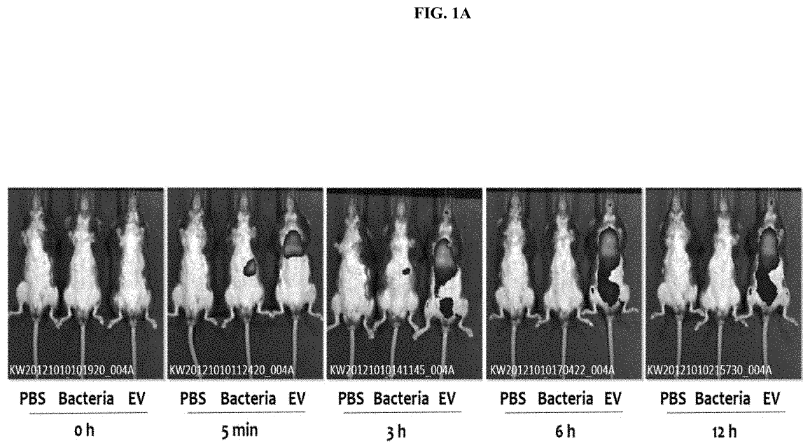

In order to evaluate whether intestinal bacteria and bacteria-derived vesicles were systemically absorbed through the gastrointestinal tract, an experiment was performed with the following method. First, a dose of 50 μg of each of fluorescence-labeled intestinal bacteria and intestinal bacteria-derived vesicles was administered through the gastrointestinal tract to the stomach of a mouse, and fluorescence was measured after 0 minute, 5 minutes, 3 hours, 6 hours, and 12 hours. As a result of observing the entire image of the mouse, as illustrated in FIG. 1A, the bacteria were not systemically absorbed, but the vesicles derived from bacteria were systemically absorbed 5 minutes after administration, and fluorescence was strongly observed in the bladder 3 hours after administration, so that it could be seen that the vesicles were excreted to the urinary tract. Further, it could be seen that the vesicles were present in the body until 12 hours after administration.

In addition, in order to evaluate the pattern in which the intestinal bacteria and the vesicles derived from the intestinal bacteria infiltrated into various organs after they were systemically absorbed, 50 μg of bacteria and vesicles derived from bacteria labeled with fluorescence were administered in the same manner as described above, and then the blood, heart, lungs, liver, kidneys, spleen, fat, and muscle were collected 12 hours after administration. As a result of observing fluorescence in the collected tissues, as illustrated in FIG. 1B, it could be seen that the vesicles derived from bacteria were distributed in the blood, heart, lungs, liver, spleen, fat, muscle, and kidneys but the bacteria were not absorbed.

### Example 2. Metagenomic Analysis of Vesicles Derived from Bacteria in Clinical Sample

After clinical samples such as blood, urine, stool, and the like was first put into a 10-ml tube and suspended matter was allowed to settle by a centrifuge (3,500×g, 10 min, 4° C.), only the supernatant was transferred to a new 10-ml tube. After bacteria and impurities were removed by using a 0.22-μm filter, they were transferred to a Centriprep tube (centrifugal filters 50 kD) and centrifuged at 1,500×g and 4° C. for 15 minutes, materials smaller than 50 kD were discarded, and the residue was concentrated to 10 ml. After bacteria and impurities were removed once again by using a 0.22-μm filter, the supernatant was discarded by using a ultra-high speed centrifugation at 150,000×g and 4° C. for 3 hours with a Type 90Ti rotor, and an aggregated pellet was dissolved in physiological saline (PBS).

Internal DNA was extracted out of the lipid by boiling 100 μl of the vesicles isolated by the above method at 100° C., and then cooled on ice for 5 minutes. And then, in order to remove the remaining suspended matter, the DNA was centrifuged at 10,000×g and 4° C. for 30 minutes, and only the supernatant was collected. And, the amount of DNA was quantified by using Nanodrop. Thereafter, in order to confirm whether the DNA derived from bacteria was present in the extracted DNA, PCR was performed with 16s rDNA primers shown in the following Table 1 and it was confirmed that genes derived from bacteria were present in the extracted genes.

TABLE 1

| | | | SEQ ID |
|---|---|---|---|
| primer | | Sequence | No. |
| 16S | 16S_V3_ | 5′- | 1 |
| rDNA | F | TCGTCGGCAGCGTCAGATGTGTATAAGA | |
| | | GACAGCCTACGGGNGGCWGCAG-3′ | |
| | 16S_V4_ | 5′- | 2 |
| | R | GTCTCGTGGGCTCGGAGATGTGTATAAG | |
| | | AGACAGGACTACHVGGGTATCTAATCC | |

The DNA extracted by the above method was amplified using the 16S rDNA primers, and then sequencing was performed (Illumina MiSeq sequencer), the results were output as a standard flowgram format (SFF) file, the SFF file was converted into a sequence file (.fasta) and a nucleotide quality score file using GS FLX software (v2.9), and then the reliability estimation for the reads was confirmed, and a portion in which the window (20 bps) average base call accuracy was less than 99% (Phred score<20) was removed. For the OTU (operational taxonomy unit) analysis, clustering was performed according to sequence similarity by using UCLUST and USEARCH, the genus, family, order, class, and phylum were clustered based on 94%, 90%, 85%, 80%, and 75% sequence similarity, respectively, classification was performed at the phylum, class, order, family, and genus levels of each OUT, and bacteria having a sequence similarity of 97% or more at the genus level were profiled by using the 16S RNA sequence database (108,453 sequences) of BLASTN and GreenGenes (QIIME).

### Example 3. Metagenomic Analysis of Bacteria-Derived Vesicles in Stool of Patient with Colon Cancer

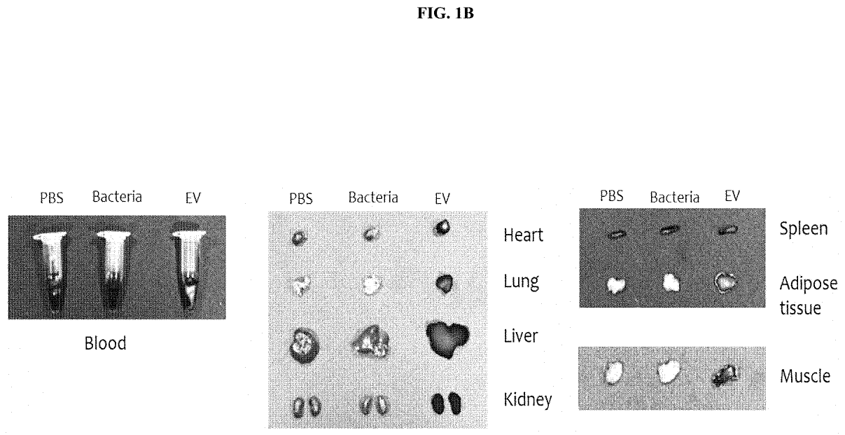

After a metagenomic analysis was performed using the method of Example 2 on the stool from 29 patients with colon cancer, and 358 normal individuals by extracting genes from vesicles present in the stool, the distribution of vesicles derived from bacteria of the genus Blautia was evaluated. As a result, it was confirmed that vesicles derived from bacteria of the genus Blautia were significantly decreased in the stool from the patients with colon cancer as compared to the stool from the normal individuals (see Table 2 and FIG. 2).

TABLE 2

| Stool | Control | | Colon cancer | | t-test | |
|---|---|---|---|---|---|---|
| Taxon | Mean | SD | Mean | SD | p-value | Ratio |
| g_Blautia | 0.0037 | 0.0100 | 0.0017 | 0.0017 | 0.0009 | 0.45 |

### Example 4. Metagenomic Analysis of Bacteria-Derived Vesicles in Blood of Patient with Liver Cancer

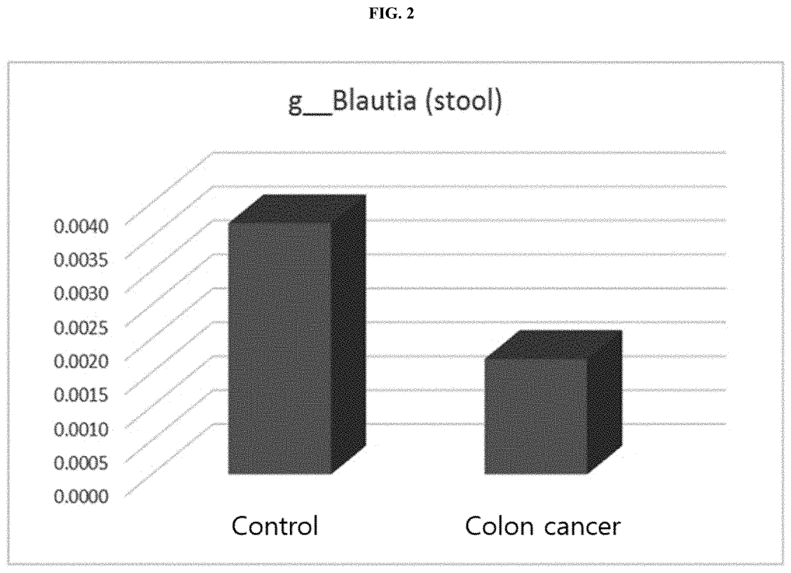

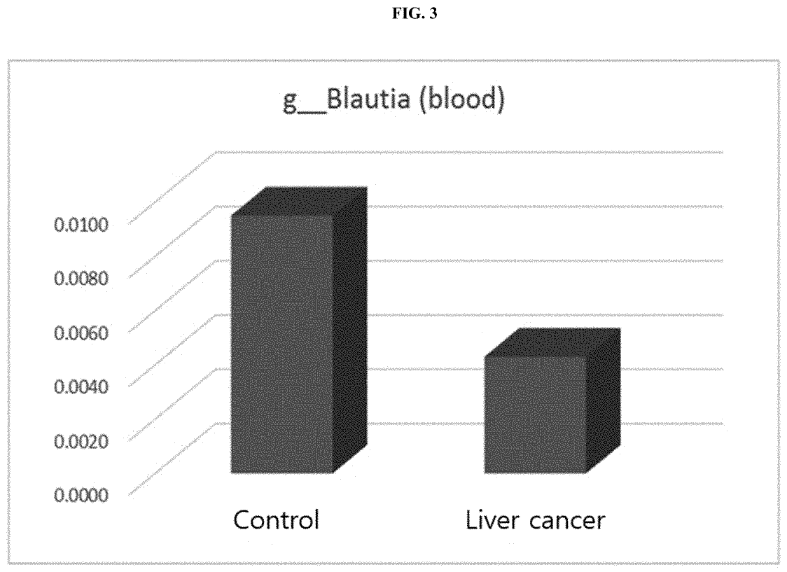

After a metagenomic analysis was performed using the method of Example 2 on the blood from 86 patients with liver cancer, and 331 normal individuals who were matched in age and sex by extracting genes from vesicles present in the blood, the distribution of vesicles derived from bacteria of the genus Blautia was evaluated. As a result, it was confirmed that vesicles derived from bacteria of the genus Blautia were significantly decreased in the blood from the patients with liver cancer as compared to the blood from the normal individuals (see Table 3 and FIG. 3).

TABLE 3

| Blood | Control | | Liver cancer | | t-test | |
|---|---|---|---|---|---|---|
| Taxon | Mean | SD | Mean | SD | p-value | Ratio |
| g_Blautia | 0.0095 | 0.0134 | 0.0043 | 0.0043 | <0.0001 | 0.45 |

### Example 5. Metagenomic Analysis of Bacteria-Derived Vesicles in Blood of Patient with Pancreatic Cancer

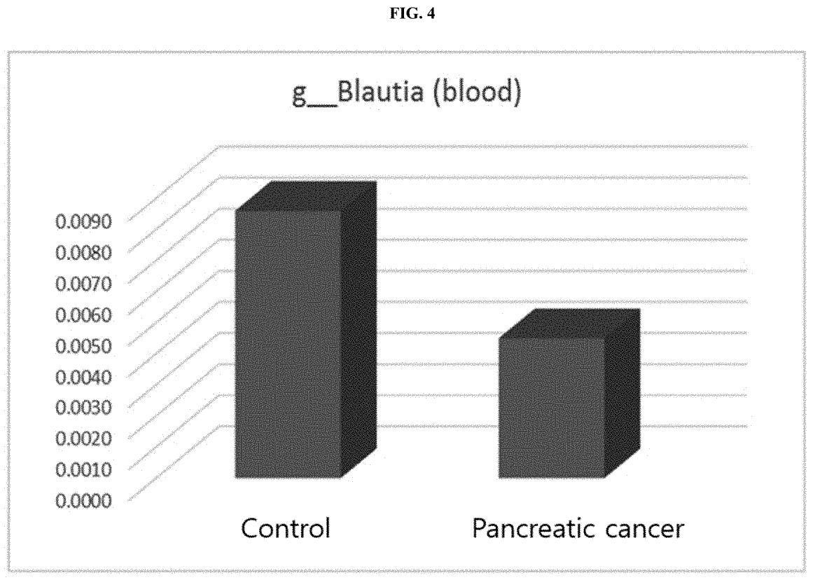

After a metagenomic analysis was performed using the method of Example 2 on the blood from 176 patients with pancreatic cancer, and 271 normal individuals who were matched in age and sex by extracting genes from vesicles present in the blood, the distribution of vesicles derived from bacteria of the genus Blautia was evaluated. As a result, it was confirmed that vesicles derived from bacteria of the genus Blautia were significantly decreased in the blood from the patients with pancreatic cancer as compared to the blood from the normal individuals (see Table 4 and FIG. 4).

TABLE 4

| Blood | Control | | Pancreatic cancer | | t-test | |
|---|---|---|---|---|---|---|
| Taxon | Mean | SD | Mean | SD | p-value | Ratio |
| g_Blautia | 0.0086 | 0.0164 | 0.0045 | 0.0071 | 0.0003 | 0.52 |

### Example 6. Metagenomic Analysis of Bacteria-Derived Vesicles in Blood of Patient with Cholangiocarcinoma

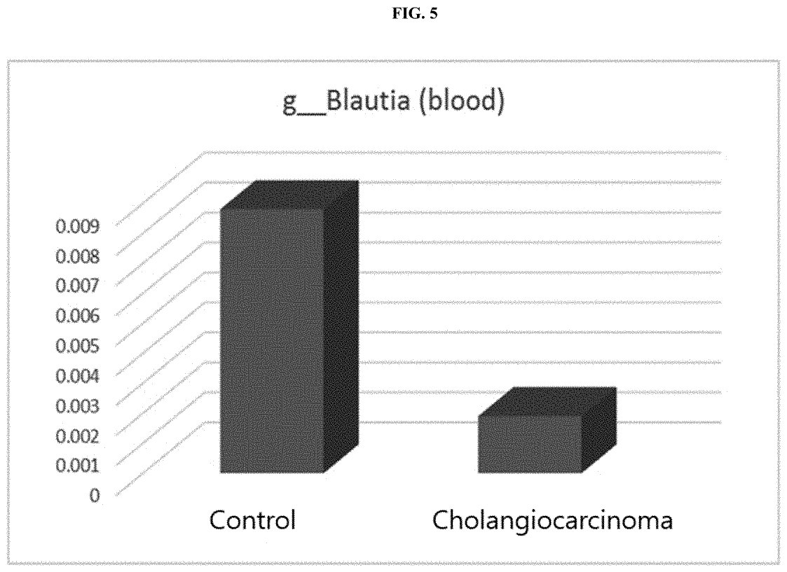

After a metagenomic analysis was performed using the method of Example 2 on the blood from 79 patients with cholangiocarcinoma, and 259 normal individuals who were matched in age and sex by extracting genes from vesicles present in the blood, the distribution of vesicles derived from bacteria of the genus Blautia was evaluated. As a result, it was confirmed that vesicles derived from bacteria of the genus Blautia were significantly decreased in the blood from the patients with cholangiocarcinoma as compared to the blood from the normal individuals (see Table 5 and FIG. 5).

TABLE 5

| Blood | Control | | Cholangiocarcinoma | | t-test | |
|---|---|---|---|---|---|---|
| Taxon | Mean | SD | Mean | SD | p-value | Ratio |
| g_Blautia | 0.0088 | 0.0169 | 0.0019 | 0.0047 | <0.0001 | 0.22 |

### Example 7. Metagenomic Analysis of Bacteria-Derived Vesicles in Blood of Patient with Ovarian Cancer

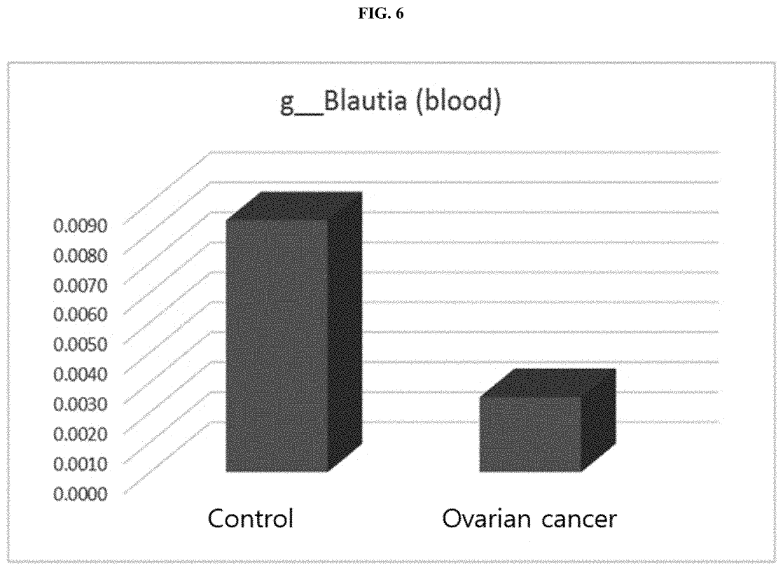

After a metagenomic analysis was performed using the method of Example 2 on the blood from 137 patients with ovarian cancer, and 139 normal individuals who were matched in age and sex by extracting genes from vesicles present in the blood, the distribution of vesicles derived from bacteria of the genus Blautia was evaluated. As a result, it was confirmed that vesicles derived from bacteria of the genus Blautia were significantly decreased in the blood from the patients with ovarian cancer as compared to the blood from the normal individuals (see Table 6 and FIG. 6).

TABLE 6

| Blood | Control | | Ovarian cancer | | t-test | |
|---|---|---|---|---|---|---|
| Taxon | Mean | SD | Mean | SD | p-value | Ratio |
| g_Blautia | 0.0084 | 0.0096 | 0.0025 | 0.0034 | <0.0001 | 0.30 |

### Example 8. Metagenomic Analysis of Bacteria-Derived Vesicles in Blood of Patient with Bladder Cancer

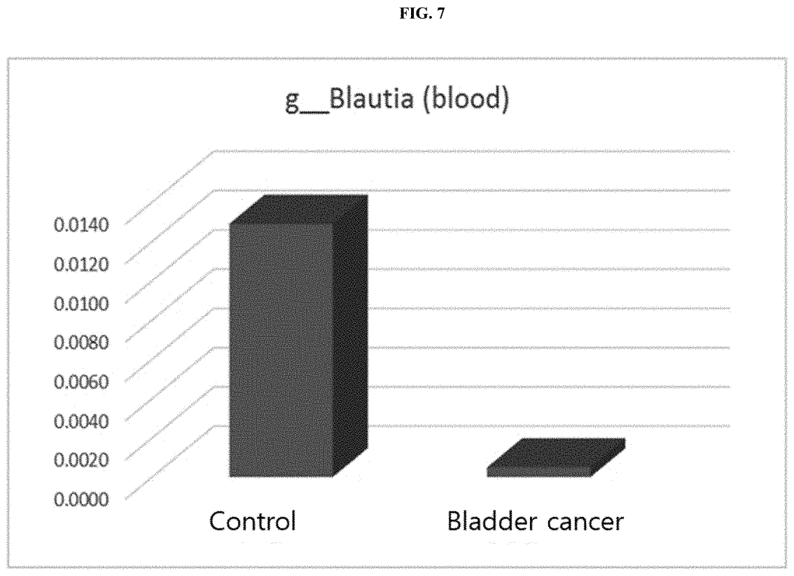

After a metagenomic analysis was performed using the method of Example 2 on the blood from 91 patients with bladder cancer, and 176 normal individuals who were matched in age and sex by extracting genes from vesicles present in the blood, the distribution of vesicles derived from bacteria of the genus Blautia was evaluated. As a result, it was confirmed that vesicles derived from bacteria of the genus Blautia were significantly decreased in the blood from the patients with bladder cancer as compared to the blood from the normal individuals (see Table 7 and FIG. 7).

TABLE 7

| Blood | Control | | Bladder cancer | | t-test | |
|---|---|---|---|---|---|---|
| Taxon | Mean | SD | Mean | SD | p-value | Ratio |
| g_Blautia | 0.0129 | 0.0209 | 0.0005 | 0.0005 | <0.0001 | 0.04 |

### Example 9. Metagenomic Analysis of Bacteria-Derived Vesicles in Blood of Patient with Myocardial Infarction

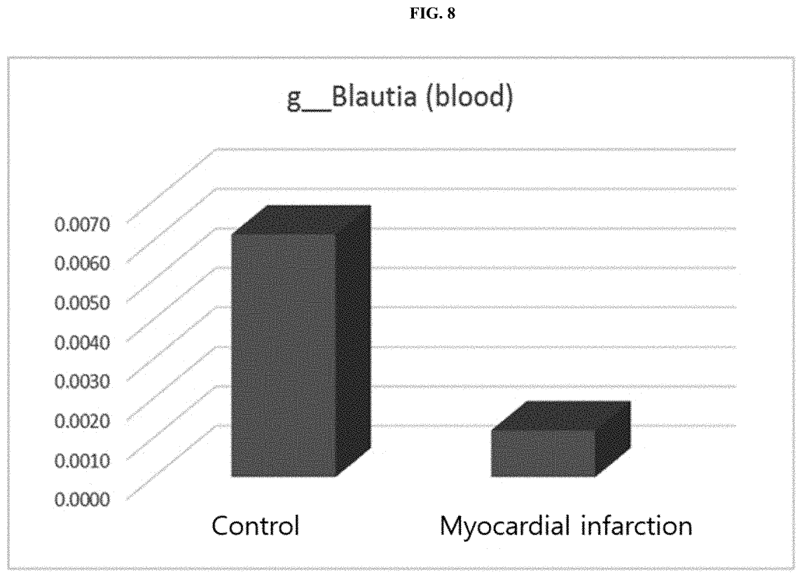

After a metagenomic analysis was performed using the method of Example 2 on the blood from 57 patients with myocardial infarction, and 163 normal individuals who were matched in age and sex by extracting genes from vesicles present in the blood, the distribution of vesicles derived from bacteria of the genus Blautia was evaluated. As a result, it was confirmed that vesicles derived from bacteria of the genus Blautia were significantly decreased in the blood from the patients with myocardial infarction as compared to the blood from the normal individuals (see Table 8 and FIG. 8).

TABLE 8

| Blood | Control | | Myocardial infarction | | t-test | |
|---|---|---|---|---|---|---|
| Taxon | Mean | SD | Mean | SD | p-value | Ratio |
| g_Blautia | 0.0061 | 0.0085 | 0.0012 | 0.0053 | <0.0001 | 0.19 |

### Example 10. Metagenomic Analysis of Bacteria-Derived Vesicles in Blood of Patient with Atrial Fibrillation

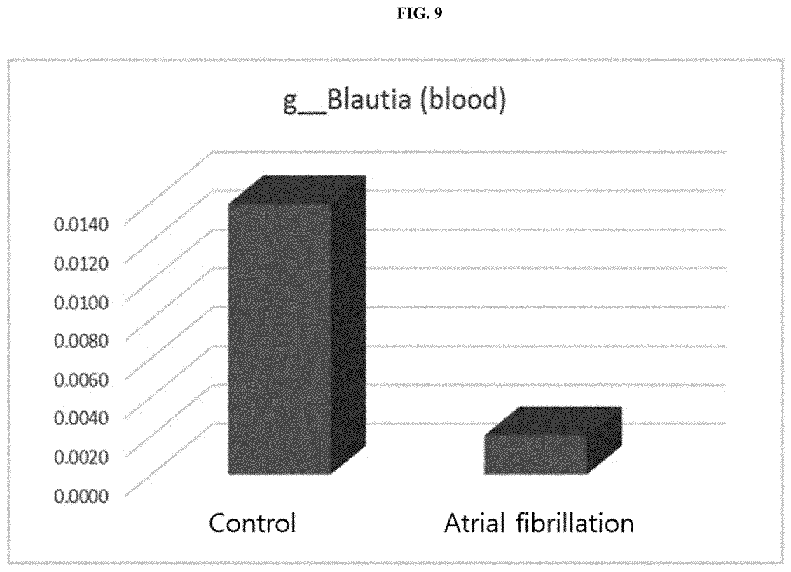

After a metagenomic analysis was performed using the method of Example 2 on the blood from 34 patients with atrial fibrillation, and 62 normal individuals who were matched in age and sex by extracting genes from vesicles present in the blood, the distribution of vesicles derived from bacteria of the genus Blautia was evaluated. As a result, it was confirmed that vesicles derived from bacteria of the genus Blautia were significantly decreased in the blood from the patients with atrial fibrillation as compared to the blood from the normal individuals (see Table 9 and FIG. 9).

TABLE 9

| Blood | Control | | Atrial fibrillation | | t-test | |
|---|---|---|---|---|---|---|
| Taxon | Mean | SD | Mean | SD | p-value | Ratio |
| g_Blautia | 0.0139 | 0.0259 | 0.0020 | 0.0028 | 0.0006 | 0.14 |

### Example 11. Metagenomic Analysis of Bacteria-Derived Vesicles in Blood of Patient with Variant Angina Pectoris

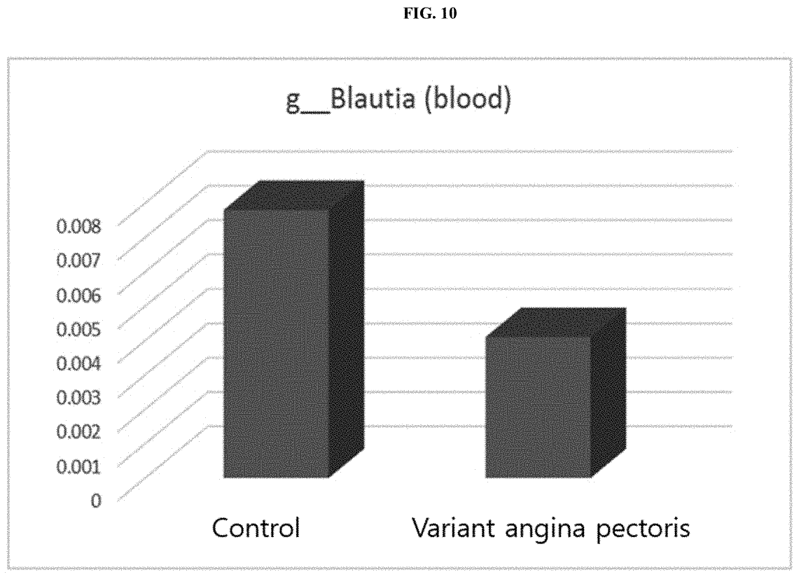

After a metagenomic analysis was performed using the method of Example 2 on the blood from 80 patients with variant angina pectoris, and 80 normal individuals who were matched in age and sex by extracting genes from vesicles present in the blood, the distribution of vesicles derived from bacteria of the genus Blautia was evaluated. As a result, it was confirmed that vesicles derived from bacteria of the genus Blautia were significantly decreased in the blood from the patients with variant angina pectoris as compared to the blood from the normal individuals (see Table 10 and FIG. 10).

TABLE 10

| Blood | Control | | Variant angina pectoris | | t-test | |
|---|---|---|---|---|---|---|
| Taxon | Mean | SD | Mean | SD | p-value | Ratio |
| g_Blautia | 0.0078 | 0.0117 | 0.0041 | 0.0049 | 0.01 | 0.53 |

### Example 12. Inflammation-Inducing Effect of Blautia coccoides-Derived Vesicles

To examine an effect of Blautia coccoides-derived vesicles on the secretion of inflammation mediators (IL-6 and TNF-α) in inflammatory cells, Raw 264.7 cells, which is a mouse macrophage cell line, were treated with the Blautia coccoides-derived vesicles at various concentrations (0.1, 1, and 10 μg/mL), followed by ELISA. More specifically, Raw 264.7 cells dispensed at 4×10⁴ cells/well in a 48-well cell culture plate were treated with various concentrations of Blautia coccoides-derived vesicles put in serum-free Dulbeco's Modified Eagle's Medium (DMEM), and cultured for 12 hours. Subsequently, the cell culture solution was collected in a 1.5 ml tube, centrifuged at 3000 g for 5 minutes to collect a supernatant. The resulting supernatant was stored at −80° C., followed by ELISA.

To perform ELISA, a capture antibody was diluted in phosphate buffered saline (PBS) and dispensed into a 96-well polystyrene plate at 50 μL/well according to a working concentration, and then allowed to react overnight at 4° C. Afterward, following washing three times with 100 μL of a 0.05% Tween-20-containing PBS (PBST) solution, 100 μL of an RD (1% bovine serum albumin (BSA)-containing PBS) solution was dispensed for blocking at room temperature for 1 hour. Samples and the standard were dispensed at 50 μL/well according to a concentration, allowed to react at room temperature for 2 hours, and washed three times with 100 μL of PBST, and then a detection antibody was diluted in RD and dispensed at 50 μL/well according to a working concentration, and then allowed to react at room temperature for 2 hours.

Subsequently, after washing three times with 100 μL of PBST, streptavidin-HRP (R&D Systems, USA) was diluted in RD at 1/40 and dispensed at 50 μL/well, and then allowed to react at room temperature for 20 minutes. Finally, after washing three times with 100 μL of PBST, 50 μL of a TMB substrate (SurModics, USA) was dispensed, and after 5 to 20 minutes, when color development progressed, a 1 M sulfuric acid solution was dispensed at 50 μL/well to stop the reaction, and then absorbance was measured at 450 nm using a SpectraMax M3 microplate reader (Molecular Devices, USA).

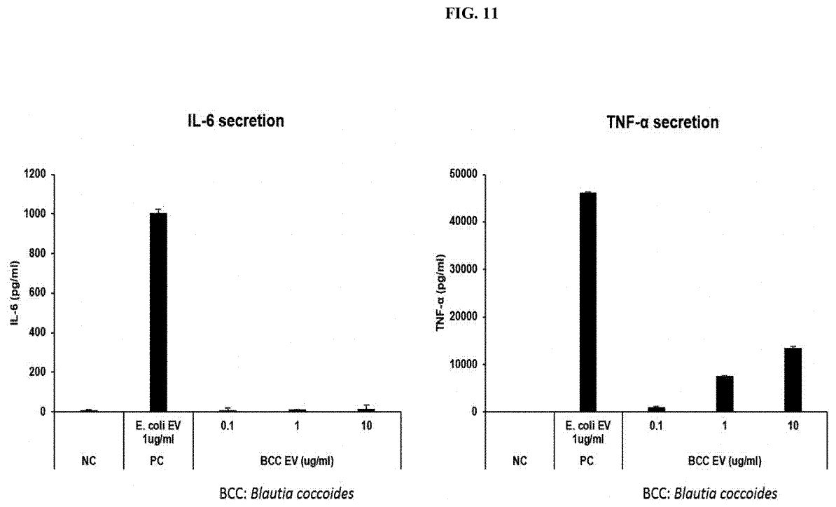

As a result, as shown in FIG. 11, when a macrophage cell line was treated with Blautia coccoides-derived vesicles, it was confirmed that the secretion of inflammation mediators was significantly lowered, compared with pathogenic vesicles such as E. coli EVs.

### Example 13. Anti-Inflammatory Effects of Blautia coccoides-Derived Vesicles

Based on the result of Example 12, to evaluate the anti-inflammatory effect of Blautia coccoides-derived vesicles, a macrophage cell line (Raw 264.7) was pre-treated with various concentrations (0.1, 1 and 10 μg/mL) of Blautia coccoides-derived vesicles for 12 hours, and then treated with 1 μg/mL of pathogenic vesicles such as E. coli EVs, and after 12 hours, the secretion levels of inflammatory cytokines were measured by ELISA. As an effective microorganism control, Lactobacillus plantarum-derived vesicles were used.

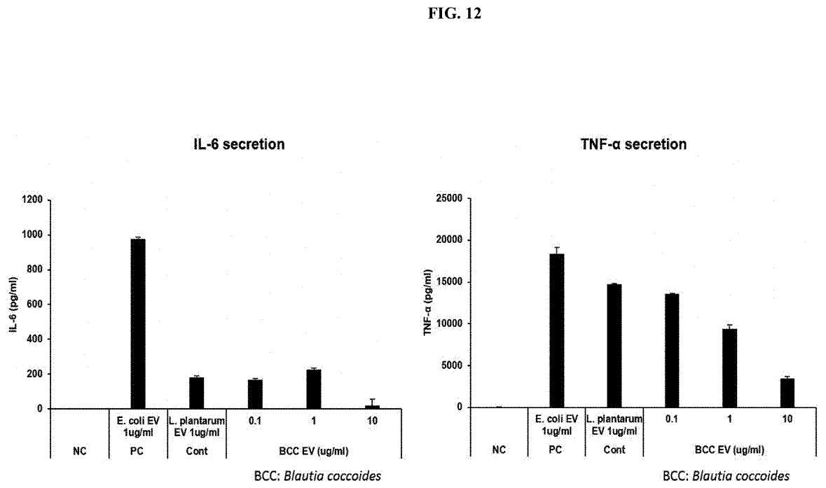

As a result, as shown in FIG. 12, when Blautia coccoides-derived vesicles were pre-treated, it was confirmed that the secretion of IL-6 and TNF-α by E. coli-derived vesicles was significantly inhibited, and particularly, the TNF-α secretion inhibitory effect caused by the pre-treatment of the Blautia coccoides-derived vesicles was significantly higher than that caused by the pre-treatment of the effective microorganism control, such as Lactobacillus plantarum-derived vesicles. This result means that inflammatory responses induced by pathogenic vesicles such as E. coli-derived vesicles can be effectively inhibited by the Blautia-derived vesicles.

The above-described description of the present invention is provided for illustrative purposes, and those of ordinary skill in the art to which the present invention pertains will understand that the present invention can be easily modified into other specific forms without changing the technical spirit or essential features of the present invention. Therefore, it should be understood that the above-described Examples are illustrative only in all aspects and are not restrictive.

### INDUSTRIAL APPLICABILITY

Since vesicles derived from bacteria of the genus Blautia according to the present invention may be used in a method of diagnosing colon cancer, liver cancer, pancreatic cancer, cholangiocarcinoma, ovarian cancer, bladder cancer, myocardial infarction, atrial fibrillation or variant angina pectoris, and a composition for preventing, alleviating or treating the disease, they are expected to be effectively used in related medical and food industry fields.

1. A method of diagnosing one or more diseases selected from the group consisting of colon cancer, liver cancer, pancreatic cancer, cholangiocarcinoma, ovarian cancer, bladder cancer, myocardial infarction, atrial fibrillation and variant angina pectoris, the method comprising the following steps:
(a) extracting DNAs from extracellular vesicles isolated from a biological samples of a normal individual and a subject;
(b) performing polymerase chain reaction (PCR) on the extracted DNA using a pair of primers prepared based on a gene sequence present in 16S rDNA to obtain each PCR product; and
(c) determining a case in which a content of extracellular vesicles derived from bacteria of the genus Blautia is lower than that of the normal individual sample, as one or more diseases selected from the group consisting of colon cancer, liver cancer, pancreatic cancer, cholangiocarcinoma, ovarian cancer, bladder cancer, myocardial infarction, atrial fibrillation and variant angina pectoris, through quantitative analysis of the PCR product.

2. The method of claim 1, wherein the biological sample in Step (a) is a blood or stool sample.

3. A method of treating one or more diseases selected from the group consisting of a colorectal disease, a liver disease, a pancreatic disease, cholangiocarcinoma, ovarian cancer, bladder cancer, myocardial infarction, atrial fibrillation, variant angina pectoris and a graft-versus-host disease, the method comprising administering to a subject in need thereof a pharmaceutical composition comprising an effective amount of extracellular vesicles derived from bacteria of the genus Blautia.

4. The method of claim 3, wherein the vesicles have an average diameter of 10 to 200 nm.

5. The method of claim 3, wherein the vesicles are secreted naturally or artificially from bacteria of the genus Blautia.

6. The method of claim 3, wherein the vesicles derived from bacteria of the genus Blautia are vesicles derived from Blautia coccoides.

7. The method of claim 3, wherein the colorectal disease is colitis, irritable bowel syndrome or colon cancer.

8. The method of claim 3, wherein the liver disease is hepatitis, liver cirrhosis or liver cancer.

9. The method of claim 3, wherein the pancreatic disease is pancreatitis or pancreatic cancer.

10. A food composition for preventing or alleviating one or more diseases selected from the group consisting of a colorectal disease, a liver disease, a pancreatic disease, cholangiocarcinoma, ovarian cancer, bladder cancer, myocardial infarction, atrial fibrillation, variant angina pectoris and a graft-versus-host disease, comprising extracellular vesicles derived from bacteria of the genus Blautia as an active ingredient.

11. The food composition of claim 10, wherein the vesicles have an average diameter of 10 to 200 nm.

12. The food composition of claim 10, wherein the vesicles are secreted naturally or artificially from bacteria of the genus Blautia.

13. The food composition of claim 10, wherein the vesicles derived from bacteria of the genus Blautia are vesicles derived from Blautia coccoides.

14. The food composition of claim 10, wherein the colorectal disease is colitis, irritable bowel syndrome or colon cancer.

15. The food composition of claim 10, wherein the liver disease is hepatitis, liver cirrhosis or liver cancer.

16. The food composition of claim 10, wherein the pancreatic disease is pancreatitis or pancreatic cancer.

17.-19. (canceled)


SEQUENCE LISTING (2 sequences)

SEQ ID NO: 1 - DNA, 50 bp, Artificial Sequence
  16S_V3_F
  misc_feature (42): a or g or c or t/u
tcgtcggcagcgtcagatgtgtataagagacagcctacgggnggcwgcag

SEQ ID NO: 2 - DNA, 55 bp, Artificial Sequence
  16S_V4_R
gtctcgtgggctcggagatgtgtataagagacaggactachvgggtatctaatcc